(19) United States
(12) Patent Application Publication
(10) Pub. No.: US 2008/0172021 A1
(43) Pub. Date: Jul. 17, 2008

(54) Apparatus and Method for Dispensing Fluid

(75) Inventor: Gerrat Dijkman, Enschede (NL)

Correspondence Address: CAHN & SAMUELS LLP, 1100 17th STREET NW, SUITE 401, WASHINGTON, DC, 20036, US

(73) Assignee: LABORIE EUROPE B.V., ENSCHEDE (NL)

(21) Appl. No.: 10/573,677

(22) PCT Filed: Sep. 24, 2004
(86) PCT No.: PCT/NL04/00663

§ 371 Date: Jul. 9, 2007

(30) Foreign Application Priority Data
Sep. 24, 2003 (NL) 1024370

(51) Int. Cl. B67D 5/08; A61B 19/00

(52) U.S. Cl. 604/403; 222/58; 222/95

(57) ABSTRACT

The invention relates to an apparatus for dispensing fluid in a dosed manner, comprising a housing, a pressure bag and connecting means, the housing comprising a carrying device for suspending the pressure bag and pumping means for pumping pressure fluid into the pressure bag, a measuring device being provided for measuring, during use, the weight or changes therein of the pressure bag with a fluid container included therein, the connecting means being designed for connecting the pressure bag to the pumping means.

The invention relates to an apparatus for dispensing fluid, in particular for medical use.

When liquid is supplied in medical applications such as drips, dripping apparatus, pumps and pressure bags can, for instance, be utilized. Such apparatus are often relatively complicated in structure and use. Moreover, with them, dosing is relatively difficult.

The object of the invention is to provide an apparatus for dispensing fluid in a dosed manner. More in particular, the object of the invention is to provide such an apparatus for dispensing medical fluid.

A further object of the invention is to provide such an apparatus with which in a simple manner a dispensing regimen can be set.

More in particular, the object of the invention is to provide such an apparatus with which fluid can be dispensed under pressure.

These and other objects are achieved with an apparatus according to the invention, characterized by the features of claim 1.

With an apparatus according to the invention, a pressure bag is provided for enclosing an at least partly flexible fluid container. Further, a housing with a carrying device is provided such that the pressure bag having a fluid container therein can be suspended. The carrying device can be designed such that the entire apparatus can be suspended therefrom but is preferably provided on the housing so that the pressure bag can be suspended therefrom. A measuring device is provided for measuring the weight of the pressure bag having a fluid container therein, or, conversely, changes in this weight. As the weight or changes therein can be measured, each time, during use, it can be determined how much fluid has been dispensed from the pressure bag, at least from this fluid container. Further, an apparatus according to the invention comprises pumping means as well as connecting means for connecting the pressure bag to the pumping means such that the pressure in the pressure bag can be raised and maintained. Preferably, regulating means are provided such as a regulating unit, with which at least the pressure in the pressure bag can be regulated, for instance be kept constant.

Preferably, the measuring device comprises a load cell with which the weight or changes therein can be measured.

In a particularly advantageous embodiment, the pumping means are designed as an electronically adjustable pump, for instance a micro pump. With it, in a particularly simple and accurate manner, each time the desired amount of pressure medium can be introduced into the pressure bag, required for correctly dispensing a fluid from a fluid container received in the pressure bag or forming part thereof.

With an apparatus according to the invention, in a particularly advantageous embodiment, a regulating unit is provided for operating components of the apparatus such as, for instance, the pumping means mentioned. Preferably, this regulating unit comprises communication means for communication with peripheral equipment, for instance for data transmission from and to the apparatus or, for instance, with a computer for programming the regulating unit. Preferably, these communication means are designed for wireless communication based on, for instance, Bluetooth® technology.

In a further particularly advantageous embodiment, an apparatus according to the invention is provided with a flow control unit, preferably operable by the regulating unit mentioned. With this flow control unit, during use, the flow of a flow of fluid emanating from an at least partly flexible fluid container provided in the pressure bag or forming part thereof can be controlled. This flow control unit can, for instance, control the same flow continually but, preferably, can also be regulated, so that a particular flow pattern can be set and constantly adjusted.

In an advantageous further embodiment, the flow control unit comprises a driving motor and a tap, while motor and tap are provided with cooperating coupling means so that with the aid of the driving motor, the tap can be operated so as to be open or closed. As a consequence of the coupling means, the tap is detachable from the unit so that it can, for instance, advantageously form part of the pressure bag, at least of the fluid container included therein. The tap can for instance be included in the dispensing tubing of the drip bag or the like and be of a disposable type.

The tap as described hereinabove can, for instance, be a ball tap or cylinder tap, provided with a rotating body with a passage having an other shape than circular so that a nonlinear relation is obtained between the available passage and the rotation of the rotating body. With it, each time, a suitable flow can be controlled while for instance changing flow conditions can be compensated simply and accurately. Moreover, with it, for instance, the accuracy with small openings can be further improved.

With such an embodiment, preferably the driving motor and regulating unit are mutually connected, for instance through wires or through wireless connecting technology, so that operating the flow control unit can be regulated by the regulating unit on the basis of, for instance, the pressure built up in the pressure bag, the amount of fluid to be dispensed per time unit, changes in the weight of the pressure bag and the fluid container mentioned and the like. Moreover, regulation can take place on the basis of a regulating profile, at least dispensing profile included in the unit.

With an apparatus according to the invention, the pressure bag is preferably of the bag-in-bag or bag-in-container type, comprising an inner volume in the form of an at least partly flexible fluid container such as a fluid bag, and an outer container provided therearound such as a bag or box. Preferably, the outer container is slightly more rigid than the fluid container such that when the pressure in the pressure bag is boosted via the connecting means as described hereinabove, pressure is applied to the fluid container and maintained. When the fluid container is opened, for instance by a needle or by opening a suitable tap, the fluid will be dispensed slightly under pressure. The pressure in the pressure bag can each time be adjusted, in order to maintain the desired pressure.

The invention moreover relates to a pressure bag, characterized by the features of claim 13.

Such a pressure bag is particularly advantageous when used in an apparatus according to the invention. The pressure bag can be reusable while the fluid container to be provided therein is preferably disposable. It can however be preferred that the pressure bag and the fluid container together be made disposable. The connecting means are then designed such that they are detachable for replacement of the pressure bag. Moreover, the earlier described tap for use in a flow unit according to the invention, which, for that matter can also be used with other dispensing apparatus, can be fixedly connected to the fluid container, at least a dispensing tube thereof, so that a disposable unit is obtained that can be closed off well before and after use.

Furthermore, the invention relates to a method for dispensing fluid, characterized by the features of claim 14.

With such a method, the advantage is achieved that a fluid can be dispensed in a particularly simple and accurate manner, for instance for a drip, for rinsing, for pressure measuring and the like. Here, both the amount of fluid dispensed and its flow can be accurately regulated, while moreover the dispensing pressure can be controlled, in combination as well as separately.

Preferably, with a method according to the invention, use is made of wireless communication technology for controlling and/or programming the apparatus to be used with it. Here, in particular Bluetooth® is advantageous.

In the further subclaims, further advantageous embodiments of the invention are described. In clarification of the invention, exemplary embodiments thereof will be further described with reference to the drawing. In the drawing:

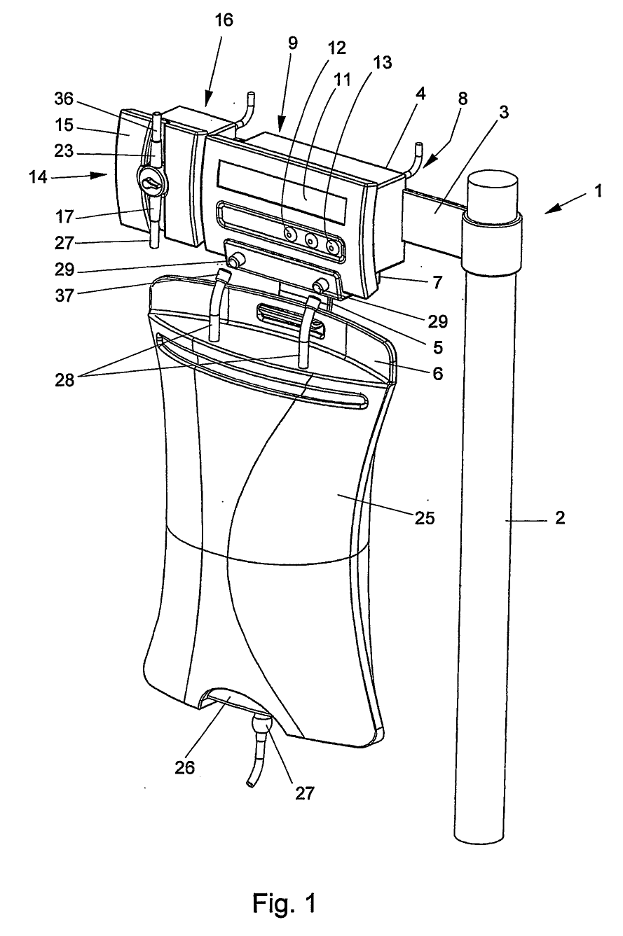

FIG. 1 shows, in perspective view, an apparatus according to the invention, in a first embodiment;

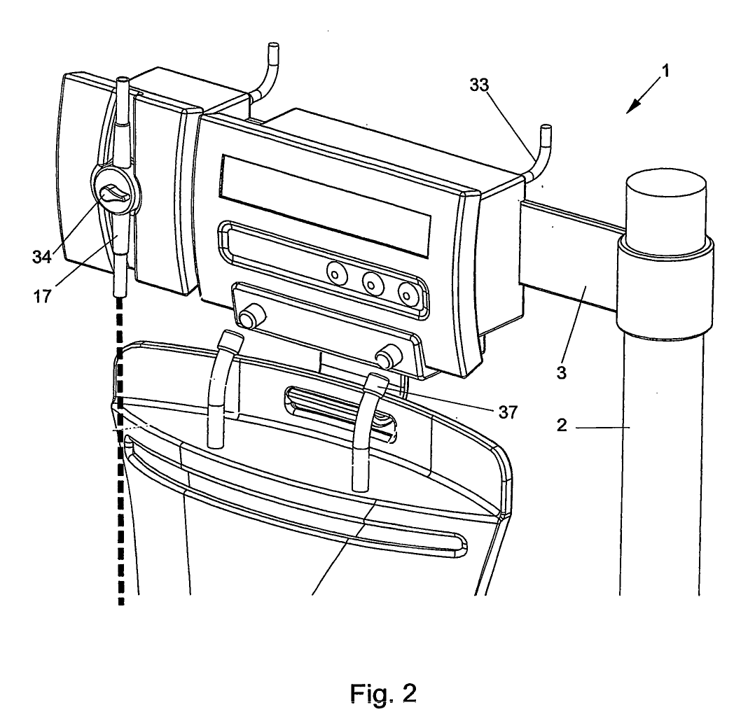

FIG. 2 shows, slightly enlarged, a part of an apparatus according to FIG. 1;

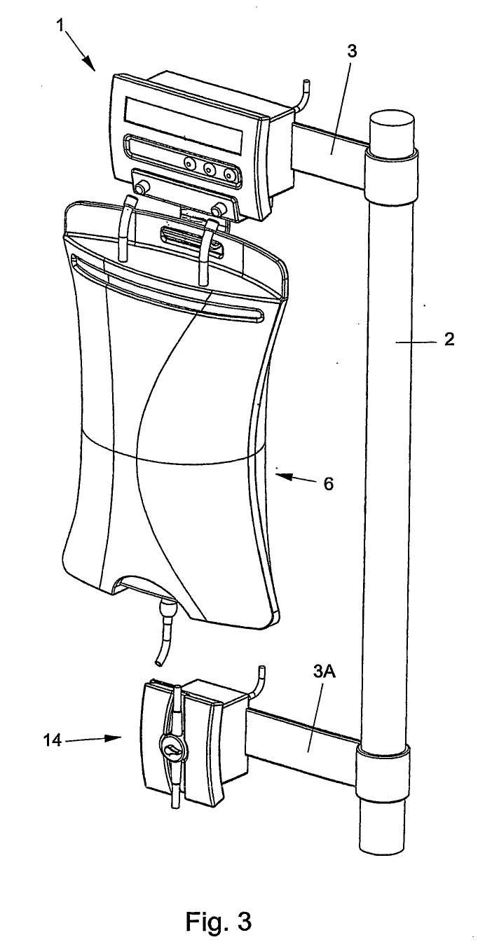

FIG. 3 shows, in perspective view, a second embodiment of an apparatus according to the invention;

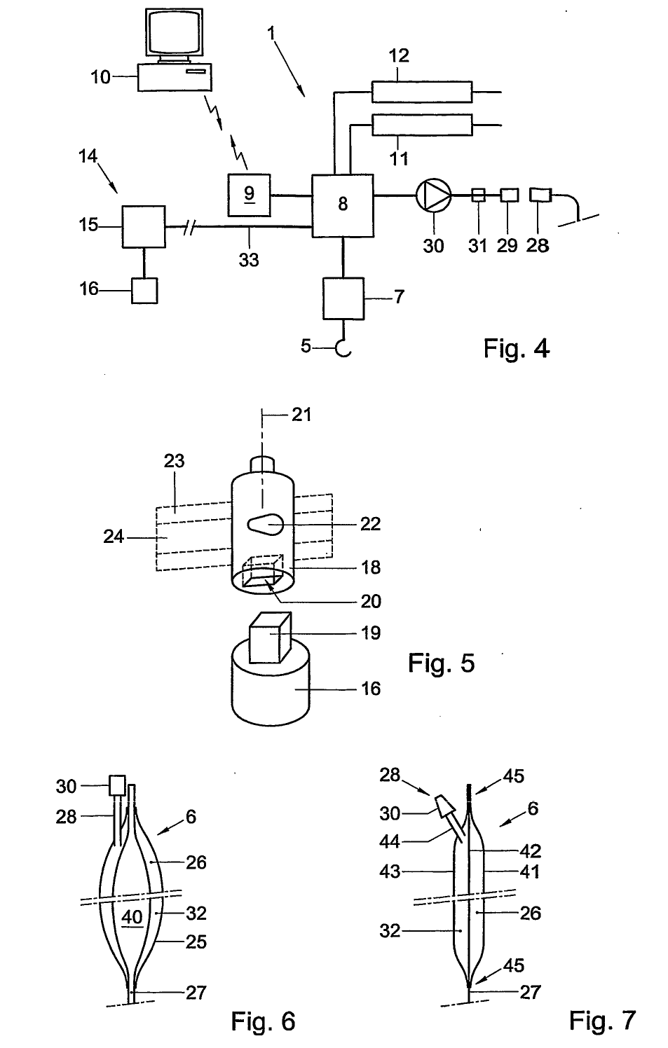

FIG. 4 schematically shows a regulating diagram of an apparatus according to the invention;

FIG. 5 shows, in perspective view, a rotation body of a tap and a driving motor for use in an apparatus, in particular a flow unit according to the invention;

FIG. 6 shows in partly cross-sectional side view a pressure bag according to the invention; and

FIG. 7 shows in partly cross-sectional side view an alternative embodiment of a pressure bag according to the invention.

In this description, identical or corresponding parts have identical or corresponding reference numerals. The exemplary embodiments shown should not be construed to be limitative in any manner and serve merely as illustration. In particular also combinations of parts of the apparatus shown fall within the framework of the invention as outlined by the claims. Unless expressly indicated otherwise, values included in this description such as dimensions, weights, positions and the like should not be construed to be limitative and not be explained strictly.

In FIG. 1, in perspective view, an embodiment of an apparatus 1 according to the invention is shown, suspended from an IV pole 2 with a bracket 3. From the bracket 3, a housing 4 is suspended forming a main unit. On this housing 4, a suspension hook 5 is provided, in FIG. 1 at the underside, from which hook 5 a pressure bag 6 is suspended. The hook 5 is attached to a load cell 7, as will be discussed hereinbelow. In the housing 4, a regulating unit it is provided, for instance an electronic connection with a battery or different feed, with which the apparatus 1 can be operated. Furthermore, in the housing 4, a wireless communication device 9, in particular of the Bluetooth® type, is provided allowing communication between the unit 8 and peripheral equipment 10 such as, for instance, a computer with which the apparatus 1 can be programmed and/or data can be read out and/or the apparatus 1 can be operated. In FIG. 1, at the front of the housing 4, a display 11 is provided as well as an interface 12 comprising operating buttons 13 with which the apparatus 1 can (also) be operated on the housing 4 itself.

On the bracket 3, further, a flow control unit 14 is attached comprising a casing 15 having a driving motor 16 therein, as well as a tap 17 which can be attached in the casing 15 to the driving motor 16 by coupling means, as shown in FIG. 5. To that end, the driving motor 16 comprises first coupling means, shown in FIG. 5 simply as a rectangular lip 19, while the rotating body 18 is provided at the underside with a recess 20 matching the lip 19 and forming the second coupling means. With this, the rotating body 18 and hence the tap 17 can be connected to the driving motor 16 and, with the aid of the motor 16, be rotated about a rotation axis 21. In the rotating body 18 a passage 21 is provided having, preferably, a noncircular cross-section. In the embodiment shown this is a somewhat egg-shaped passage. In the embodiment shown, the tap 17 is a ball tap, at least a cylinder tap type, the passage being regulated through rotation of the rotating body 18 relative to a tap body 23, schematically represented in FIG. 5 in interrupted lines. This body 23 comprises, for instance, a cylindrical passage 24 while the passage 22 can be pivoted wholly or partly in front of it or away from it. Such taps are sufficiently known per se. With the tap 17 according to the invention, the passage 22 has a non-circular cross section while through rotation of the body 18 about the axis 21, a non-linear relation is obtained between the rotation and the passage surface. Thus, particularly accurate regulation can take place, also with small flow rates.

The pressure bag 6 comprises a rigid outer casing 25 with an at least partly flexible fluid container 26 included therein, in particular a fluid bag defining a closed off volume and provided at the underside, in a customary manner, with a discharge tube 27 connected to the tap 17. From the tap 17, a further tubing 36 extends to, for instance, an IV needle, catheter or the like. In the embodiment shown in FIG. 1, the outer casing 25 is double-walled, while the outer wall is relatively rigid and is provided on the inside, in a customary manner, with an inflatable wall. Preferably, the fluid container 26 is disposable. At the top side of the pressure bag shown in FIG. 1, two connections 28 are provided ending up in the space within the double wall of the outer wall 25, and which can be connected to second connections 29 on the housing 4, which second connections 29 are in communication with a pump 30 within the housing 4. Through operation of the pump 13 with the aid of the regulating unit 8, with it, a pressure fluid can be introduced via the connecting means 28, 29 (shown separately in FIG. 1) into the pressure bag 6, in the double wall 25, so that the fluid container 6 is compressed. With the aid of the regulating unit 8, the pump 30 can each time be operated such that the pressure in the fluid container 26 is always kept at a desired value, for instance always equal. The pump 30 can then be used as measuring instrument for measuring the pressure, but a separate pressure meter 31 can be included too.

In FIG. 6, in cross-sectional side view, schematically, a pressure bag 6 according to the invention is shown in an alternative embodiment. Here, a bag-shaped fluid container 26 is provided, enclosed by a second, somewhat bag-shaped outer container 25 designed so as to be rigid relative to the fluid container. Between the fluid container 26 and the outer container 25, a pressure space 32 is provided in which the connecting means 28 end up. At the underside, the tube 27 is attached in a customary manner or is an integral part of the pressure bag 6. This is a bag-in-bag or a bag-in-box type pressure bag. Preferably, this is a disposable. The connecting means 28 and 29 can be provided with specific compatible couplings 37, designed such that only compatible pressure bags 6 can be used. In this manner, the safety is considerably increased.

In FIG. 7, a further alternative embodiment of the pressure bag according to the invention is shown, preferably of disposable design. Here, three sheets 41, 42, 43 of plastic are sealed onto each other by their longitudinal edges 45. Preferably, the sheets 41, 42, 43 are rectangular. Between two sheets 41, 42 the fluid container 26 is formed, at least a compartment for the fluid 40. Between the middle sheet 42 and the other adjoining sheet 43, the pressure space 32 is provided. In sheet 43, a connection 28, 29 is provided in the form of a valve 44 through which pressure fluid can be introduced into the pressure space 32. In a manner known per se the fluid compartment can be connected to the tube 27 or be equipped therewith.

In FIG. 2, in slightly enlarged condition, a part of an apparatus 1 according to the invention is shown, while a part of an electric connection 33 is included between the regulating unit 8 and the flow control unit 14 such that the flow control 14 can be operated by the regulating unit 8. At the front of the tap 17 for that matter, a button 34 is provided so that it can also be operated manually.

In FIG. 3, an apparatus 1 according to invention is shown in an alternative embodiment with the flow control unit 14 suspended below the pressure bag 6 on a second bracket 3A. Thus, the distance between the fluid container 26 and the flow control unit 14 is limited and the tube 27 needs not first be guided upwards before it proceeds to the patient. It is preferred for that matter that the connection 33 is also designed so as to be wireless.

In FIG. 4, a regulating diagram of an apparatus 1 according to the invention is shown as earlier described. With such an apparatus, the dispensing of fluid can be dosed as follows.

With the aid of the communication means 9, in particular the Bluetooth®, the regulating unit 8 can be programmed to a particular patient and a selected pressure bag. For instance the amount of fluid to be dispensed per time unit can be determined, a desired flow pattern can be set and calibrations can be carried out. A pressure bag 6 with fluid container 26 having a fluid 40 to be dispensed therein is suspended from the hook 5 and the connecting means 28, 29 are coupled to the coupling means 37. The tap 17 which is preferably fixedly included in the tube 27 is placed on the motor 16 with the aid of the coupling means 19, 20 and the discharge tube 36 from the tap 17 is connected to a patient via, for instance, an IV needle (not shown). The apparatus 1 can be switched on, for instance with the aid of one of the buttons 13, so that with the aid of the pumping means 30, the pressure bag 6 is brought to a desired pressure and fluid 40 is dispensed. The flow of the fluid is controlled with the aid of the flow control unit 14, in particular the motor 16, operated by the regulating unit 8.

On the display 11, for instance the amount of dispensed fluid, the type of fluid, the time duration, the flow and the like can be read. As wireless communication means are provided, in particular BlueTooth®, data can also be read on peripheral equipment, for instance a computer 10 that needs not be in the same room as the apparatus 1. Various apparatus 1 can simply be regulated and checked in this manner.

In the exemplary embodiments shown in FIGS. 1-3, the pressure bag 6 is each time suspended from the hook 5, which hook is connected to the load cell 7 or a different measuring device for measuring the weight, at least changes therein, of the pressure bag with fluid container. However, the hook 5 can also be provided on the housing 4 such that it can be suspended from the bracket 3 with the aid of the hook 5. Naturally, then, the weight of the housing 4 has to be compensated. The fluid container 26 can for instance have a content of 0.5, 2 or 3 litres.

The invention is not limited in any manner to the exemplary embodiments represented in the description and the drawings. Many variations are possible within the framework of the invention as outlined by the claims. For instance, other communication means can be provided for operating the apparatus while this can, moreover, be designed to be set manually. The pressure bag can be provided with pressure measuring means that can be connected to the regulating unit 8, for instance via the coupling means 28, so that each pressure bag itself is provided with measuring means. Optionally, the flow control unit 14 can be integrated in the housing 4 while, naturally, the apparatus 1 can also be suspended in another manner or even be laid down. The pressure bag can then for instance be placed on a load cell, serving as a pair of scales. In such a manner too, with an apparatus 1 according to the invention, dispensing of fluid can be dosed and checked in a simple manner. If so desired, the pumping means 30 can also be set such that with them, the fluid 40 in the pressure bag 6, at least in the fluid container 26, can for instance be put and held in motion continuously or periodically. Further, a tap 17 for use within the invention itself can be provided with driving means such as a driving motor which can be placed in a matching flow control unit provided with, for instance, an electric connecting means 4 for such a motor. As a result, for each pressure bag a specific motor can be provided with, for instance, preset dosing regimes.

These and many comparable variations are understood to fall within the framework of the invention as outlined by the claims.

1. An apparatus for dispensing fluid in a dosed manner, comprising:
a housing,
a pressure bag having a fluid container therein, wherein the housing includes a carrying device for suspending the pressure bag,
pump means for pumping pressure fluid into the pressure bag,
a measuring device for measuring, during use, the weight and/or changes in the weight of the pressure bag, and
connecting means being designed for connecting the pressure bag to the pumping means.

2. An apparatus according to claim 1, wherein the pumping means comprises an electronically operated pump, in particular a micro pump.

3. An apparatus according to claim 1, wherein the measuring device comprises a load cell from which, during use, either the pressure bag is suspended or the apparatus is suspended.

4. An apparatus according to claim 1, wherein a regulating unit is provided for operating components of the apparatus, in particular at least the pumping means, preferably at least on the basis of data coming from the measuring device.

5. A apparatus according to claim 4, wherein the regulating unit comprises communication means for communication with peripheral equipment, in particular for wireless communication, more in particular Blue Tooth® means.

6. An apparatus according to claim 4, wherein the regulating unit comprises a measuring device for measuring pressure in the pressure bag, in particular via the connecting means, and comprises regulating means for regulating the pressure in the pressure bag through operation of the pumping means.

7. An apparatus according to claim 4, wherein further a flow control unit is provided which can be operated by the regulating unit.

8. An apparatus according to claim 7, wherein the flow control unit comprises a driving motor, provided with first coupling means, and a tap, provided with second coupling means, while, during use, the tap can be coupled to the driving motor via the first and second coupling means such that the tap can be opened and closed and in particular be set with the aid of said driving motor.

9. An apparatus according to claim 8, wherein the driving motor and the regulating unit are electronically interconnected for operation of the driving motor by the regulating unit.

10. An apparatus according to claim 8, wherein the tap is included in the tubing which extends from a fluid container included, during use, in the pressure bag.

11. An apparatus according to claim 8, wherein the tap has a noncircular passage.

12. An apparatus according to claim 1, wherein the pressure bag is a bag-in-a-bag or bag-in-container type, in which an inner volume is provided in which fluid is included that can be dispensed, while between an inner wall and an outer wall a second volume is provided, connected to the connecting means for building up pressure in the pressure bag.

13. A pressure bag, provided with an at least partly flexible fluid container and a pressure vessel extending therearound such that the pressure bag is of the bag-in-bag or bag-in-container type, the fluid container being provided with a connecting element for dispensing fluid therefrom and the pressure vessel being provided with connecting means for connection to a source for pressure fluid and therewith pressurizing the fluid container.

14. A method for dispensing fluid from an at least partly flexible fluid container, which fluid container is included in or forms part of a pressure bag, wherein the weight of the pressure bag having the fluid container therein is measured continuously or periodically while the fluid container is kept pressurized through supply of a pressure fluid in the pressure bag, while with the aid of a regulating unit, the pressure and the dispending of fluid is checked based on the measured weight and/or changes therein and a profile stored in the regulating unit or supplied thereto, while the discharge rate of fluid from the fluid container is controlled with the aid of a flow control unit in a dispensing tube.

15. A method according to claim 14, wherein the regulating unit is operated and/or programmed with the aid of wireless communication technology such as BlueTooth® technology.
